SEQUENCE LISTING (9 sequences)

SEQ ID NO: 1 - protein, 10 aa, Artificial Sequence
  E7 56-65 peptide from immunodominant epitope region of Human Papilloma Virus 16 E7 protein
TFCCKCDSTL

SEQ ID NO: 2 - protein, 10 aa, Artificial Sequence
  E7 57-66 peptide from immunodominant epitope region of Human Papilloma Virus 16 E7 protein
FCCKCDSTLR

SEQ ID NO: 3 - protein, 10 aa, Artificial Sequence
  E7 58-67 peptide from immunodominant epitope region of Human Papilloma Virus 16 E7 protein
CCKCDSTLRL

SEQ ID NO: 4 - protein, 10 aa, Artificial Sequence
  E7 59-68 peptide from immunodominant epitope region of Human Papilloma Virus 16 E7 protein
CKCDSTLRLC

SEQ ID NO: 5 - protein, 10 aa, Artificial Sequence
  E7 60-69 peptide from immunodominant epitope region of Human Papilloma Virus 16 E7 protein
KCDSTLRLCV

SEQ ID NO: 6 - protein, 10 aa, Artificial Sequence
  E7 61-70 peptide from immunodominant epitope region of Human Papilloma Virus 16 E7 protein
CDSTLRLCVQ

SEQ ID NO: 7 - protein, 11 aa, Artificial Sequence
  E7 58-68 peptide from immunodominant epitope region of Human Papilloma Virus 16 E7 protein
CCKCDSTLRLC

SEQ ID NO: 8 - protein, 25 aa, Artificial Sequence
  E7 46-70 peptide from immunodominant epitope region of Human Papilloma Virus 16 E7 protein
EPDRAHYNIVTFCCKCDSTLRLCVQ

SEQ ID NO: 9 - protein, 9 aa, Artificial Sequence
  E7 79-87 control peptide from immunodominant epitope region of Human Papilloma Virus 16 E7 protein
LEDLLMGTL

(19) United States
(12) Patent Application Publication
(10) Pub. No.: US 2009/0117140 A1
(43) Pub. Date: May 7, 2009

(54) Human papilloma virus dominant CD4 T cell epitopes and uses thereof

(76) Inventors: Mayumi Nakagawa, Little Rock, AR (US); Alessandro D. Santin, Little Rock, AR (US)

Correspondence Address: Benjamin Aaron Adler;ADLER & ASSOCIATES, 8011Candle Lane, Houston, TX, 77071, US

(21) Appl. No.: 12/284,704

(22) Filed: Sep. 24, 2008

Related U.S. Application Data
(60) Provisional application No. 60/995,307, filed on Sep. 26, 2007.

(51) Int. Cl. A61K 39/12; C12Q 1/02; C07K 7/00; A61K 38/08; A61K 38/16; C07K 14/00; A61K 35/12

(52) U.S. Cl. 424/186.1; 435/29; 424/93.7; 530/328; 530/324; 514/15; 514/13

(57) ABSTRACT

Provided herein are methods of determining immunodominant T cell epitopes within a protein expressed in an individual and immunotherapy directed towards a protein in an individual using these determined epitopes. The method comprises administering autologous dendritic cells pulsed with a recombinant protein to the individual, establishing T-cell lines therefrom and incubating the T cell lines with representative peptides from the protein to measure and identify those peptides from the protein inducing the T cell response. Also provided are synthetic or recombinant peptides or immunogenic compositions thereof comprising the identified peptide(s) or peptides of similar sequence and a method of preventing or treating a pathophysiological condition.

### CROSS-REFERENCE TO RELATED APPLICATION

This non-provisional application claims benefit of provisional application U.S. Ser. No. 60/995,307 filed on Sep. 26, 2007, now abandoned.

### FEDERAL FUNDING LEGEND

This invention was produced using funds from Federal government under grant no. NCI R21CA094507 from the National Institutes of Health. Accordingly, the Federal government has certain rights in this invention.

### BACKGROUND OF THE INVENTION

1. Field of the Invention

The present invention relates generally to the field of immunology. More specifically, the present invention involves identification of dominant CD4 T cell epitopes in the human Papilloma virus proteins and its use in treating cancer such as cervical cancer.

2. Description of the Related Art

Cervical cancer is the second most common malignancy among women worldwide (1) with 400,000 new cases being diagnosed annually (2). Annually 12,000 to 14,000 new cases of squamous cell cancer of the cervix are reported in the United States (3), resulting in about 3,500 deaths per year. The link between human Papilloma virus and the development of cervical cancer is well known. Among the over one hundred different types of Human Papilloma virus, at least 15 are strongly associated with invasive squamous cell cancer of the cervix (4). High risk Human Papilloma virus, the most commonly HPV 16 and HPV 18, encode two viral E6 and E7 oncoproteins. They are constitutively expressed in cervical cancer cells and are required for malignant transformation as well as maintenance of malignant phenotype of cervical cancer (5-6).

Human Papilloma virus infection is also associated with the precursor lesion of cervical cancer, squamous intraepithelial lesion (7-12). While most low-grade squamous intraepithelial lesions prospectively regress spontaneously (13-14), some progress to high-grade squamous intraepithelial lesions. These high-grade lesions, in particular, cervical intraepithelial neoplasia 3 is associated with a high rate progression to invasive cervical cancer (Nash 15-16).

Two early gene products, E6 and E7, mediate transformation to a malignant phenotype by Human Papilloma virus. Both of these viral proteins have been shown to interact with the products of cellular human tumor suppressor genes. The E7 protein, a well-characterized cytoplasmic/nuclear protein with little intratypic sequence variation is important in the vaccination against Human Papilloma virus-containing cervical cancer. HPV 16 and 18 are associated with vast majority of cervical cancers and it is known the E7 oncoprotein is important in the induction and maintenance of cellular transformation in most Human Papilloma virus-containing cancers. Thus, the E7 protein from HPV16 and HPV18 is a significant target antigen in the vaccination of cervical cancer. However, all the studies reported so far have demonstrated the usefulness of full-length E7 protein in the vaccination against such cancer. One such study reported that autologous dendritic cells pulsed with the recombinant, full-length E7 protein elicited a specific CD8+ cytotoxic T lymphocyte response against autologous tumor target cells in patients with HPV16 or HPV18-positive cervical cancer and induced CD4+ T cell proliferative response (17).

Cell-mediated immunity has been shown to play an important role in controlling Human Papilloma virus infection and Human Papilloma virus-associated diseases. CD4 T-cells are important in the development of anti-tumor responses (18-21). It is believed that the effectiveness of these CD4 T-cells lies in their ability to deliver help for priming and maintaining CD8 cytotoxic T lymphocytes, which are thought to serve as the dominant effector cells in tumor elimination. Immunohistochemical analyses of squamous intraepithelial lesions and cervical cancer specimens have demonstrated the presence of activated cytotoxic T lymphocytes in lesions (22). The CD4 T-cells activate cytotoxic T lymphocytes by producing T helper 1 cytokines and by providing activation signals for priming of tumor-specific cytotoxic T lymphocytes to professional antigen presenting cells (23-26).

Antigen presenting cells, which may transfer peripheral antigenic signals to the lymphoid organs, play a crucial role in the induction of antigen-specific T-cell immunity responses to Human Papilloma virus infection and Human Papilloma virus-associated tumors. Dendritic cells as professional antigen presenting cells express high level of major histocompatibility complex and co-stimulatory molecules. Insufficient or improper activation of dendritic cells, caused by lack of pro-inflammatory signal, leading to antigen presentation not in an appropriate co-stimulatory context is one reason for the failure of antitumor immunity.

Vaccination with autologous, tumor antigen loaded properly activated dendritic cells in vitro present promising immunotherapy modality for tumors. With the development of techniques for dendritic cell isolation, antigen loading and maturation, dendritic cell-based vaccines have obtained rapid and remarkable progress in recent decade (27-28).

After successful induction of Human Papilloma virus-specific T-cell responses in vitro, several clinical trials were carried out targeting different stage cervical cancer using autologous monocyte-derived, recombinant HPV 16/18 E7 oncoprotein-loaded dendritic cells, and promising clinical responses were demonstrated in some patients (29-30). Although dendritic cells pulsed with E7 protein can induce systemic B and T cell responses in cervical cancer patients with recurrent disease refractory to standard treatment modalities, immunosuppression induced by pretreatment with chemotherapy and radiotherapy may impose limitations on the efficacy of active vaccination strategies in late stage cervical cancer patients (30).

Thus, the prior art is deficient in peptide antigens, derived from the Human Papilloma virus E7 protein that have been identified based on the T cell responses, to be used as sources of antigens for therapeutic vaccines or for dendritic cell immunotherapy to treat cervical cancers. The present invention fulfills this long-standing need and desire in the art.

### SUMMARY OF THE INVENTION

The present invention is directed to a method of determining a pattern of immunodominant T cell epitopes within a protein in an individual. This method comprises administration of autologous dendritic cells pulsed with a recombinant protein to an individual. T-cell lines from the individual are then established. The established T cell lines are then incubated with peptides representative of the protein and the specific T cell response in the incubated cells is then measured. The peptides that induce a T cell response are identified, wherein the sequence of the peptides corresponds to a region within the protein. Thus, the immunodominant T cell epitopes within the protein are determined.

The present invention is directed to a related method of immunotherapy directed towards a protein in an individual. This method comprises isolating immune cells from the individual and incubating the isolated immune cells with peptides comprising one or more of immunodominant T cell epitopes identified using the method described supra. These incubated immune cells are transferred back to the individual, where the immune cells activate a specific immune response in the individual, thereby generating immunotherapy targeted towards the protein in the individual.

The present invention also is directed to synthetic peptides having sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

The present invention is direct further to synthetic peptides, that are at least 80%, and up to and including about 90% similar in composition, to one or more sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

The present invention is directed further still to an immunogenic composition comprising one or more recombinant or synthetic peptides of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8 or one or more peptides that are at least 80%, and up to and including about 90% similar in composition to these sequences.

The present invention is directed further still to a related method of preventing or treating a pathophysiological condition involving expression of a protein in an individual. Such a method comprises administering the immunogenic composition identified herein, where the composition activates specific immune response in the individual, thereby treating the pathophysiological condition in the individual.

### BRIEF DESCRIPTION OF THE DRAWINGS

The following figures form part of the present specification and are included to further demonstrate certain aspects of the present invention. The invention may be better understood by reference to one or more of these figures in combination with the detailed description of specific embodiments presented herein.

FIGS. 1A-1C shows results of enzyme-linked immunospot assays performed to screen-positive T-cell clones from subject 15-04 with 15-mer peptides in the E7 46-70 and E7 61-85 regions. A total of 1,000 T-clone cells were plated in duplicate or in triplicate, along with 1×10⁵ autologous Epstein-Barr Virus-transformed B-lymphoblastoid cell line cells. In FIG. 1A, three (#11, #45, #207) of six screen-positive T-cell clones showed specificity with E7 56-70. In FIG. 1B, three (#304, #329, #349) of four screen-positive T-cell clones showed specificity with E7 56-70. In FIG. 1C, two screen-positive T-cell clones in the E7 61-85 region were negative upon re-testing and demonstrated to be false-positive.

FIGS. 2A-2B shows results of enzyme-linked immunospot assays performed to assess the mode of antigen presentation. The HPV 16 E7 epitope appears to be processed through the major histocompatibility complex class II antigen processing pathway in which the E7 protein is presented exogenously to dendritic cells. In FIG. 2A, one thousand T-clone cells were plated along with 105 autologous Epstein-Barr Virus-transformed B-lymphoblastoid cell line cells infected with vaccinia virus expressing E6, vaccinia virus expressing E7, and wild-type vaccinia virus, respectively, at a multiplicity of infection of 5. Six tested T-cell clones showed negative response with vaccinia virus expressing E6, vaccinia virus expressing E7, and wild-type vaccinia virus. In FIG. 2B, one thousand T-clone cells and 5×10⁴ autologous Epstein-Barr Virus-transformed B-lymphoblastoid cell line cells were plated along with 2.5×10³ autologous dendritic cells pulsed with E7 56-70 peptide, with HPV 16 E7 protein, or neither. Three T-cell clones tested demonstrated positive responses with the E7 56-70 peptide and the E7 protein-pulsed dendritic cells, but not with dendritic cells alone.

FIGS. 3A-3C show core epitope sequences within the E7 56-70 region. The core sequence of this novel HPV 16 E7 CD4 epitope appears to be E7 58-68 (11-mer). In FIG. 3A, E7 58-67 (10-mer) followed by E7 59-68 (10-mer) showed the highest number of spot-forming units among the overlapping 10-mer peptides tested (#11, #207, and #304); One thousand T-clone cells were plated along with 1×10⁵ autologous Epstein-Barr Virus-transformed B-lymphoblastoid cell line cells. The peptide concentration was 10 μM. A representative example of two experiments is shown. In FIG. 3B, E7 56-70 (15-mer), E7 58-68 (11-mer), E7 58-67 (10-mer), and E7 59-68 (10-mer) demonstrated equally high number of spot-forming units but not E7 58-66 (9-mer), E7 59-67 (9-mer), and E7 60-68 (9-mer); one thousand T-clone cells were plated along with 10⁵ autologous Epstein-Barr Virus-transformed B-lymphoblastoid cell line cells. The peptide concentration was 10 μM. FIG. 3C, E7 58-68 (11-mer), E7 58-67 (10-mer), and E7 59-68 (10-mer) were serially diluted and showed equivalent numbers of spot-forming units. One thousand T-clone cells were plated along with 5×10⁴ autologous Epstein-Barr Virus-transformed B-lymphoblastoid cell line cells. A representative result (#207) of two T-cell clones (#45 and #207) tested is shown.

FIG. 4 shows the restriction element for the novel CD4 T-cell epitope is the Human Leukocyte Antigen-DR 17 molecule using an enzyme linked immunospot assay. One thousand T-clone cells were plated along with 10⁵ Epstein-Barr Virus-transformed B-lymphoblastoid cell line cells matching at the designated Human Leukocyte Antigen major histocompatibility complex class II types. A representative example from three experiments performed is shown. * represents autologous Epstein-Barr Virus-transformed B-lymphoblastoid cell line cells.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. Some embodiments of the invention may consist of or consist essentially of one or more elements, method steps, and/or methods of the invention. It is contemplated that any method or composition described herein can be implemented with respect to any other method or composition described herein.

As used herein, the term “immunologically effective amount” refers to an amount that results in an improvement or remediation of the symptoms of the disease or condition due to induction of an immune response. Those of skill in the art understand that the effective amount may improve the patient's or subject's condition, but may not be a complete cure of the disease and/or condition. For example, an immunologically effective amount of an optimal vaccine incorporates tumor-specific cytotoxic T lymphocytes epitopes and tumor-specific helper T cell epitopes to induce activation of both tumor-specific cytotoxic CD8 T-cells and helper CD4 T-cells, which are critical to establish ideal antitumor immunity.

The present invention examines the pattern of CD4 T cell epitopes in the HPV16 or HPV 18 E7 protein recognized by T lymphocytes from women with cervical cancer who have been vaccinated with autologous dendritic cells pulsed with recombinant full-length HPV 16 or 18 E7 protein, and their T lymphocytes were isolated and cultured in vitro. The T lymphocytes were incubated with peptides representative of the E7 protein and enzyme-linked immunospot assay was performed to identify regions within the E7 protein, which contained the dominant as well as subdominant epitopes. T cell clones with the specificity to the dominant epitope were isolated on the basis of interferon-γ secretion. The restriction element necessary for T cell epitope recognition was identified.

Four out of the eight subjects vaccinated showed the presence of potential T-cell epitopes. The dominant epitope, i.e. forming the greatest number of spot-forming units, was found in E7 amino acid region 46-70 for all subjects. The present invention further defined the minimum and optimal amino acid sequence of dominant epitopes of HPV type 16 E7 46-70 (EPDRAHYNIVTFCCKCDSTLRLCVQ; SEQ ID NO: 8) restricted by Human Leukocyte Antigen major histocompatibility complex class II molecule and CD4 T-cell epitope E7 58-68 (CCKCDSTLRLC; SEQ ID NO: 7) restricted by the Human Leukocyte Antigen-DR17 molecule.

Only a few of Human Papilloma virus-specific CD4 T-cell epitopes have been described for the E7 protein. These include E7 50-62 restricted by Human Leukocyte Antigen-DR15, E7 43-77 restricted by Human Leukocyte Antigen-DR3, and E7 35-50 restricted by Human Leukocyte Antigen-DQ2 (31); and E7 61-80 restricted by Human Leukocyte Antigen-DR9 (32). Additionally, it has been shown that T-cell responses to a Human Leukocyte Antigen-DQB1*02-restricted HPV 16 E7 71-85 epitope (15 amino acids long) correlates with spontaneous regression of high-grade squamous intraepithelial lesions in HPV 16-positive women (33). Recently, one HPV 16 E6 CD4 epitope, i.e., E6 127-141 restricted by Human Leukocyte Antigen-DRB1*01, and one HPV 18 E6 CD4 epitope, i.e., E6 43-57 restricted by Human Leukocyte Antigen-DRB1*15, were described (34).

Thus, considerable effort has been made to identify antigenic epitopes of Human Papilloma virus. In the present invention, a novel CD4 T-cell epitope HPV 16 E7 58-68 restricted by the Human Leukocyte Antigen-DR17 molecule has been identified. Computer algorithms would not have predicted the presence of this epitope since it does not contain known binding motifs (HIV Human Leukocyte Antigen Anchor Residue Motif Scan). However, the present invention differs from the previous efforts to identify antigenic epitopes. First, the approach taken in the present invention had an advantage of being able to select T cell epitopes based on magnitude of the T cell response. Hence, these epitopes may play a significant role in viral clearance. Second, the present invention studied women with cervical cancer immunized with autologous dendritic cells expressing Human Papilloma virus peptides and the identified immunodominant CD4 T cell epitopes specific for E7 peptides are likely important given that the Human Leukocyte Antigen types of the women are different (Table 1). Analysis of more vaccine recipients is needed to extend this observation. Third, the approach of the present invention identifies T cell epitopes that would not have been predicted using computer model algorithms. Taken together, the HPV 16 E6 46-70 region is an immunodominant region in which numerous T-cell epitopes are contained.

TABLE 1
Human Leukocyte Antigen alleles of four subjects from which
T-cell lines containing potential T-cell epitopes

| | HUMAN LEUKOCYTE ANTIGEN | |
|---|---|---|
| Subject | Class I | Class II |
| 5-02 | A*2901, A*3301, | DRB*1301 (DR13), DRB*1401 (DR14), DRB3*0101 |
| | B*5701, B*3501, | (DR52), |
| | Cw*0401 | DRB3*0202 (DR52), DQB1*0503 (DQ5), DQB1*0603 |
| | | (DQ6) |
| 10-01 | A*0201, A*0301, | DR4, DR12, DQ3, DRw52, DRw53 |
| | B*1501, B*4402, | |
| | Cw*0304, Cw*0501. | |
| 15-03 | A*0201, A*0301, | DRB1*0301 (DR17), DRB1*1501 (DR15), DRB3*0101 |
| | B*0702, B*0801, | (DR52), |
| | Cw*0304, Cw*0701. | DRB5*0101 (DR51), DQB1*0201 (DQ2), DQB1*0602 |
| | | (DQ6) |
| 15-04 | A*0101, A*3201, | DRB1*0301 (DR17), DRB1*1501 (DR15), DRB3*0101 |
| | B*0702, B*0801, | (DR52), |
| | Cw*0701, Cw*0702 | DRB5*0101 (DR51), DQB1*0201 (DQ2), DQB1*0602 |
| | | (DQ6) |

A more classical approach was utilized to identify Human Papilloma virus E7 epitopes through the generation of T cell lines by in vitro stimulation of CD8 cells with autologous dendritic cells infected with vaccinia virus expressing E6 and vaccinia virus expressing E7, and a limiting dilution assay to isolate a T cell clone before defining the peptide sequence and its associated restriction element (35). However, this method is impractical to identify antigenic epitopes of pathogens which are expected to generate a small number of circulating T lymphocytes and thus fail to identify the Human Papilloma virus epitope due to the low frequency of T-cell clones.

The method in the present invention used to identify the Human Papilloma virus E7 46-70 epitope incorporated key technical advances, which make it feasible to identify new epitopes even when particular T lymphocytes with the specificity might be scarce. These advances included (i) using overlapping 15-mer peptides covering the entire protein to identify the region in which the epitope is contained; (ii) magnetically selecting for interferon-γ-secreting peptide-specific T lymphocytes and (iii) seeding autologous and allogeneic Epstein-Barr Virus-transformed B-lymphoblastoid cell line cells for the enzyme-linked immunospot assay, reducing the number of T cell clone cells required to 1000 cells per well.

Based on the observations above it is contemplated that Human Papilloma virus specific-CD4 T cell epitopes can be identified in women being treated for squamous intraepithelial lesions. Cervical swab samples are collected from women with abnormal pap smears for Human Papilloma virus DNA testing. The ones that test positive for HPV16 will have to undergo phlebotomy. The patterns of their CD4 T cell epitopes contained in the HPV16 E6 and E7 proteins are examined. The minimal and optimal amino acid sequences of these epitopes are defined along with the restricting Human Leukocyte Antigen molecules.

It is further contemplated that these defined epitopes are sources of antigen for dendritic cell immunotherapy. The epitopes are assessed by examining their expression on primary tumor cell lines derived from cervical cancer. The broadness of the utility of these epitopes is further examined by cross-presentation and cross-recognition of analogous CD4 and CD8 T cell epitopes from HPV16 variants and other high-risk Human Papilloma virus types.

Generally, such methods can be performed on an individual who is diagnosed with a pathophysiological condition, is in remission, or is diagnosed with a precursor of the pathophysiological condition. Examples of such pathophysiological conditions include, but are not limited to a neoplastic disease or disorder, an autoimmune disease or disorder or a pathogen-related disease. Further, examples of the neoplastic disease include but are not limited to prostate cancer, ovarian cancer, or cervical cancer. In the case of cervical cancer, the individual might have been previously infected with Human Papilloma virus, had abnormal pap smear results, or had been diagnosed with precursor of cervical cancer for example, squamous intraepithelial lesion.

Although the present invention used the method to identify immunodominant epitopes of Human Papilloma virus protein, this method can be used to identify one or more dominant epitopes of any protein, such as prostate specific antigen or cancer antigen-125, as long as the protein can be cloned into a recombinant virus that can infect dendritic cells. Therefore, this method can be used to identify epitopes from many other pathogens and self-antigens. Additionally, it is further contemplated to be used to identify immunodominant epitopes of proteins of Human Papilloma viruses other than HPV16 and other than E6 and E7 proteins; for instance, proteins such as E1, E2, E4, E5, L1 or L2. The T cells in these methods are stimulated with autologous dendritic cells infected with recombinant vaccinia virus expressing the entire Human Papilloma virus protein. This Human Papilloma virus protein is an E6, an E7, an E2, an E4, an E5, a L1, or a L2 protein. The Human Papilloma virus protein belongs to any of the following type of Human Papilloma virus: 16, 18, 31, 33, 35, 39, 45, 51, 52, 56, 58, 59, 68, 73 or other high-risk types. The specific T cell response in these methods is determined by the enzyme-linked immunospot assay.

In one embodiment of the present invention there is provided a method of determining immunodominant T cell epitopes within a protein expressed in an individual, comprising: administering autologous dendritic cells pulsed with a recombinant protein to the individual, establishing T cell lines from the individual; incubating the stimulated T cell line with peptides representative of the protein; measuring the specific T cell response in the incubated cells; and identifying peptides that induce T cell response, wherein the sequence of the peptide corresponds to a region within the protein, thereby determining the immunodominant T cell epitopes within the protein in the individual. This method may further comprise determining an amino acid sequence of the immunodominant T cell epitopes identified using the method described supra.

The individual benefiting from such a method may include, but is not limited to, one who is diagnosed with a pathophysiological condition, is in remission, or is diagnosed with a precursor of the pathophysiological condition. Specifically, the pathophysiological condition may include but is not limited to, a neoplastic disease or disorder, an autoimmune disease or disorder or a pathogen-related infection or disease. Examples of the neoplastic disease or disorder may include but are not limited to Human Papilloma virus infection, atypical squamous cells of undetermined significance, squamous intraepithelial lesion, cervical intraepithelial lesion, cervical cancer, prostate cancer, ovarian cancer, vulvar cancer, anal cancer, head cancer, neck cancer or other types of cancer. Generally, the T-cell epitopes identified by this method are CD4 T-cell epitopes or CD8 T-cell epitopes. Further, the sequences of the peptides comprising the immunodominant T cell epitopes in Human Papilloma virus protein have amino acid sequences SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8 (Table 2); or have amino acid sequences of these peptides comprising at least 80% and up to and including 90% similarity of the composition of the immunodominant T cell epitopes in Human Papilloma virus protein of amino acid sequences SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8 (Table 2).

Furthermore, the immunodominant T cell epitopes are contained in a 25 amino acid residue peptide comprising the immunodominant T cell epitopes of HPV protein which has an amino acid sequence of SEQ ID NO: 8. Additionally, the immunodominant T cell epitope identified by this method may be an 11 amino acid residue long peptide. In the Human Papilloma virus protein, such an epitope may have the sequence of SEQ ID NO: 7 or have at least 80% similarity and up to and including a 90% similarity to SEQ ID NO:7 or SEQ ID NO: 8.

TABLE 2

Peptide sequences covering T cell epitopes
regions identified as immunodominant.

| | | |
|---|---|---|
| SEQ | | |
| ID | HPV 16 | |
| NO | E7 PEPTIDE REGION | PEPTIDE AMINO ACID SEQUENCE |

| | | |
|---|---|---|
| 1 | E7 56-65 | TFCCKCDSTL |
| 2 | E7 57-66 | FCCKCDSTLR |
| 3 | E7 58-67 | CCKCDSTLRL |
| 4 | E7 59-68 | CKCDSTLRLC |
| 5 | E7 60-69 | KCDSTLRLCV |
| 6 | E7 61-70 | CDSTLRLCVQ |
| 7 | E7 58-68 | CCKCDSTLRLC |
| 8 | E7 46-70 | EPDRAHYNIVTFCCKCDSTLRLCVQ |

In another embodiment of the present invention there is provided a method of immunotherapy towards a protein in an individual, comprising isolating immune cells from the individual; incubating the isolated immune cells with peptide comprising one or more than one immunodominant T cell epitopes identified using the method described supra; and transferring the incubated immune cells back to the individual, where the immune cells produce a specific immune response in the individual, thereby generating immunotherapy towards the protein in the individual.

The protein in such a method may be a Human Papilloma virus E6 or E7 protein. The immune cells used in this method are T cells or dendritic cells. The individual likely to benefit from this immunotherapy may include, but is not limited to, one who has a positive Human Papilloma virus DNA test, abnormal pap smear results, or who has been diagnosed with a Human papillomavirus infection, a precursor of cervical cancer for example, squamous intraepithelial lesion, or who has been diagnosed with cervical cancer, or is suspected of or at risk of suffering from the disease. Since antigenic epitopes for many other pathogens and self antigens can be identified using the method described in the present invention, the immunotherapy described above will benefit individuals suffering from other cancers, pathogen-related diseases and autoimmune diseases.

In yet another embodiment of the present invention there is provided synthetic peptides comprising sequences of peptides shown in SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

In a related embodiment there is provided synthetic peptides comprising sequences of peptides with at least 80% sequence similarity and up to and including a 90% similarity to the sequences with SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

In yet another embodiment of the present invention there is provided a an immunogenic composition comprising one or more recombinant or synthetic peptides having the sequence of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8 or an immunogenic composition comprising at least 80% sequence similarity and up to and including a 90% similarity to the synthetic peptides described supra. Such an immunogenic composition may further comprise an adjuvant. Such a sequence or sequences may be expressed in a recombinant viral vector or a plasmid or may be a chemically synthesized peptide using well-known and established molecular biological and/or chemical synthetic techniques.

In yet another embodiment of the present invention there is provided a method of preventing or treating a pathophysiological condition involving expression of protein in an individual. Such a method comprises administering the immunologically effective amounts of the immunogenic composition described herein to the individual, where the composition activates a specific immune response in the individual, thereby preventing or treating the pathophysiological condition in the individual.

Generally, the individual who might benefit from this method is one who has abnormal pap smear results, has been diagnosed with a precursor of cervical cancer such as squamous intraepithelial lesion or is suspected or at risk of suffering from cervical cancer. The cancer prevented or treated using such a method may include but is not limited to Human Papilloma virus-positive cancers. As discussed herein, since this method can be used to identify immunodominant epitopes of proteins other than Human Papilloma virus that are expressed in other diseases or disorders, these diseases or disorders can also be treated in the manner analogous to the treatment directed towards Human Papilloma virus protein.

The immunogenic composition disclosed herein may be administered either alone or in combination with another drug or a compound. Such a drug or a compound may be administered concurrently or sequentially with the immunogenic composition. The effect of co-administration with the immunogenic composition is to lower the dosage of the drug or the compound normally required that is known to have at least a minimal pharmacological or therapeutic effect against the disease that is being treated. Concomitantly, toxicity of the drug or the compound to normal cells, tissues and organs is reduced without reducing, ameliorating, eliminating or otherwise interfering with any cytotoxic, cytostatic, apoptotic or other killing or inhibitory therapeutic effect of the drug or the compound.

The composition described herein and the drug or compound may be administered independently, either systemically or locally, by any method standard in the art, for example, subcutaneously, parenterally, intraperitoneally, intradermally, intramuscularly, topically, nasally, or by inhalation spray, by drug pump or contained within transdermal patch or an implant. Dosage formulations of the composition described herein may comprise conventional non-toxic, physiologically or pharmaceutically acceptable carriers, or vehicles suitable for the method of administration.

The immunogenic composition described herein and the drug or compound may be administered independently one or more times to achieve, maintain or improve upon a therapeutic effect. It is well within the skill of an artisan to determine dosage or whether a suitable dosage of either or both of the immunogenic composition and the drug or compound comprises a single administered dose or multiple administered doses.

As is well known in the art, a specific dose level of such an immunogenic composition for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

The following examples are given for the purpose of illustrating various embodiments of the invention and are not meant to limit the present invention in any fashion.

### EXAMPLE 1

### Subjects and T Cell Lines

Ten female patients diagnosed with stage IB or IIA cervical cancer (nine HPV 16-positive, one HPV 18-positive) were treated with HPV 16 or 18 E7-pulsed dendritic cells vaccinations in a dose-escalation phase I clinical trial. Each participant received five subcutaneous injections, and they were well tolerated. Detailed descriptions of the ten subjects with stage IB-IIA cervical cancer who participated in the phase I escalating dose trial and the methods used to generate the T-cell lines have been described. At the time of study participation, the subjects had no evidence of disease recurrence after radical surgery. The University of Arkansas approved the protocol for the Medical Sciences Internal Review Board and the Food and Drug Administration. Written informed consent was obtained from each participant. Seventeen T-cell lines established from peripheral blood mononuclear cells collected after vaccine administrations were available from all ten subjects for this study. These peripheral blood mononuclear cells were collected on day 56 (referred to as time 1) after three vaccine administration, on day 98 after five vaccine administrations (time 2), or on day 144 (without any further vaccinations; time 3). The T-cell lines were established by stimulating peripheral blood mononuclear cells with autologous mature dendritic cells pulsed with recombinant full-length HPV 16 or 18 E7 protein as described. These T-cell lines were cryopreserved until one day prior to performing interferon-γ enzyme-linked immunospot assays.

### EXAMPLE 2

### Autologous Dendritic Cell Production Antigen Presentation Immunotherapy

Units of buffy coat from blood donors with known Human Leukocyte Antigen types (A, B, C, DQ, DR) are obtained from Lifeblood Biological Services (Memphis, Tenn.). These buffy coat units are shipped via FedEx using an overnight service. Peripheral blood mononuclear cells will be isolated from the buffy coat using the Ficoll Hypaque (Amersham Biosciences, Piscataway, N.J.) density gradient method. Monocytes (CD14⁺) are isolated from peripheral blood mononuclear cells by positive selection using CD14 microbeads (Miltenyi Biotec, Auburn, Calif.), following the manufacturer's instructions. Autologous dendritic cells are established by growing monocytes in the presence of granulocyte-macrophage colony-stimulating factor (50 ng/mL) and recombinant IL-4 (100 U/mL) for 6 days.

### EXAMPLE 3

### Synthetic HPV 16 or 18 E7 Peptides

A set of 15-mer peptides overlapping by the central 10 amino acids and a set of 9-mer peptides overlapping by the central 8 amino acids for the HPV 16 E7 protein have been described (36). A set of 15-mer peptides, (also overlapping by 10 amino acids) covering the HPV18 E7 protein, was synthesized by CPC Scientific, Inc. (San Jose, Calif.). To define the core sequence of the T-cell epitope from subject 15-04, six 10-mer peptides HPV E7 56-65 (TFCCKCDSTL, SEQ ID NO: 1); HPV 16 E7 57-66 (FCCKCDSTLR, SEQ ID NO: 2); HPV 16 E7 58-67 (CCKCDSTLRL, SEQ ID NO: 3); HPV 16 E7 59-68 (CKCDSTLRLC, SEQ ID NO: 4); HPV 16 E7 60-69 (KCDSTLRLCV, SEQ ID NO: 5); HPV 16 E7 61-70 (CDSTLRLCVQ, SEQ ID NO: 6) and one 11-mer peptide HPV 16 E7 58-68 (CCKCDSTLRLC, SEQ ID NO: 7) which includes the sequences of the two positive 10-mer peptides were also synthesized (CPC Scientific, Inc.).

### EXAMPLE 4

### Identifying Regions that Contain Potential T-Cell Epitopes

To identify regions that contain potential T-cell epitopes, an interferon-γ enzyme-linked immunospot assay was performed utilizing a method used to study CD8 T-cell epitopes (37). In this study, unselected T-cell lines were examined using peptide pools (three 15-mer peptides contained in each pool) covering the HPV 16 or HPV 18 E7 protein depending on the Human Papilloma virus type of the subject's tumor. MultiScreen 96-wells plate (Millipore, Bedford, Mass.) was coated with 50 μl/well of mouse anti-interferon-γ monoclonal antibody (1-DIK; Mabtech, Stockholm, Sweden) at a concentration of 5 μg/ml and incubated overnight at 4° C. The coated wells were washed 4 times with phosphate-buffered saline and blocked with 50 μl/well RPMI 1640 medium supplemented with 5% heat-inactivated pooled human serum. Then the plate was incubated in a humidified 37° C., 5% CO₂ incubator for 1 hr. A total of 1×10⁵ T-cells/well containing 20 U/ml of recombinant human interleukin-2 were added along with peptides pools (10 μM of each peptide) in duplicate or triplicate depending on the cell numbers. Negative control wells contained media only, and positive control wells contained 10 μg/ml phytohemagglutin.

Following 24 hr incubation in a humidified 37° C., 5% CO₂ incubator, the wells were washed four times with phosphate-buffered saline containing 0.05% Tween-20 and 50 μl secondary antibody (biotin-conjugated anti-interferon-γ monoclonal antibody; 7-B6-1, Mabtech) at a concentration of 1 μg/ml was added to each well. After 2 hr of incubation at 37° C., the wells were washed four times with phosphate-buffered saline containing 0.1% Tween-20, and 50 μl of Avidin-bound biotinylated horseradish peroxidase H (Vectastain Elite Kit; Vector laboratories, Inc., Burlingame, Calif.), prepared according to the manufacturer's instructions, was added to each well and the plate was incubated for 2 hr at 37° C. The plate was washed four times with phosphate-buffered saline containing 0.1% Tween-20 and 50 μl of stable diaminobenzene (Research Genetics, Huntsville, Ala.) was added to each well and incubated for 5 min to develop the reaction at room temperature. After the wells were washed three times with deionized water and air-dried, spot-forming units were counted using an automated Elispot analyzer (Cell Technology, Inc., Jessup, Md.). HPV 16 E7-specific T-lymphocyte response was considered positive if spot-forming units in peptide-containing wells are at least two times above the corresponding negative control wells (37-38).

### EXAMPLE 5

### Selecting and Growing Antigen-Specific T-Cell Clones

Using a method to characterize HPV-specific CD8 T-cell epitopes (36, 39) antigen-specific T-cell clones were isolated and characterized. Sufficient cells from the T-cell lines, which contained potential T-cell epitope, were remaining only for subjects 15-03 and 15-04 to perform this part of the analysis. Briefly, the T-cell lines were stimulated with 10 μM of each peptide contained in the positive peptide pools and positively selected with the use of the interferon-γ Secretion Assay Cell Enrichment and Detection Kit (Miltenyi Biontec INC., Auburn, Calif.) following manufacturer's instructions. Selected interferon-γ positive cells were plated at a concentration of 0.5 cell/well in the presence of 0.5× feeder-cell mixture (Yssel's medium containing 1% pooled human serum, penicillin G 100 U/ml, streptomycin 100 μg/ml, 5×10⁵/ml irradiated allogeneic peripheral blood mononuclear cells, 5×10⁴/ml irradiated JY cells, 0.1 μg/ml PHA). On day 5, 100 μl of Yssel's medium containing recombinant human IL-2 (20 u/ml) was added to each well. After identification of growing microcultures, the cells were transferred to a well of 24-well plates containing 1 ml of a 1× feeder-cell mixture (Yessel's medium containing 1% pooled human serum, penicillin G 100 U/ml, streptomycin 100 μg/ml, 1×10⁶/ml irradiated allogeneic PBMC, 1×10⁵/ml irradiated JY cells, 0.1 μg/ml phytohemagglutinin). The limiting dilution method described here is a commonly used method of isolating T-cell clones. However, monoclonality was not established since molecular analysis of the T-cell clones was not performed.

### EXAMPLE 6

### Epstein-Barr Virus-Transformed B-Lymphoblastoid Cell Line Cells

An Epstein-Barr virus-transformed B-lymphoblastoid cell line is established (40), for each subject that CD4 T-cell epitopes is characterized. In short, CD3- and CD14-depleted peripheral blood mononuclear cells are incubated, with occasional mixing, for 90 minutes with a supernatant fluid of B958 containing free Epstein-Barr virus virions. Ninety percent of Epstein-Barr virus virions are removed by centrifugation, and the peripheral blood mononuclear cells are grown in RPMI 1640 containing 10% fetal calf serum, penicillin G (1,000 U/mL), streptomycin (1,000 μg/mL), and cyclosporin A. The peripheral blood mononuclear cells will be incubated and monitored for growth of characteristic cell clumps. Once a stable Epstein-Barr virus-transformed B-lymphoblastoid cell line is established, it is cryopreserved to prevent loss due to contamination. Epstein-Barr virus-transformed B-lymphoblastoid cell line cells are utilized to reduce the number of T-cell clones necessary to define the minimal/optimal amino acid sequences and the restriction element of the T-cell epitope.

### EXAMPLE 7

### Chromium Release Assay

Either the cells of the autologous Epstein-Barr virus transformed B lymphobastoid cell line or of the allogenic Epstein-Barr virus-transformed B lymphoblastoid cell line sharing Human Leukocyte Antigen class 1 molecule(s) with subjects were pulsed with 10 μM of peptide antigen. The cells were radiolabeled with 200 μCi sodium chromate (Na₂⁵¹CrO₄) and incubated with the peptide. After washing, the cells were plated in triplicate in 96-well plates at 3×10³ cells per well. Effector cells were added at eight different effector:target cell ratios. The plated cells were pelleted by centrifugation and then incubated for 5 h at 37° C. in a humidified 5% CO₂ incubator. The supernatants were harvested using a Skatron harvesting press and the chromium-51 was counted using a gamma counter (Packard Instruments, Meriden, Conn.). Percent specific lysis was calculated as described (41).

### EXAMPLE 8

### Screening T-Cell Clones

The enzyme-linked immunospot assay plate was coated and blocked as described above. Ninety-four T-cell clones were tested at a time by plating them (50 ml of culture medium containing the T-cell clones per well) in the same well position of three separate enzyme-linked immunospot assay plates for subject 15-04. One hundred thousand autologous Epstein-Barr Virus-transformed B-lymphoblastoid cell line cells were also added to all wells. A 15-mer peptide pool (10 mM each) covering the HPV 16 E7 46-70 region was added to the first plate, a 15-mer peptide pool (10 mM each) covering the HPV 16 E7 61-85 region was added to the second plate, and media alone was added to the third plate. One well of each plate was used as a no T-cell clone negative control; one well was used as a phytohemagglutinin-positive control. The plate was incubated and developed as described above. The wells that show spots in an enzyme-linked immunospot assay plate with one peptide pool, but not in other plates, were considered to potentially contain T-cell clones with the specificity of interest.

### EXAMPLE 9

### Confirming the Specificity of T-Cell Clones

T-cell clones that were positive in the screening enzyme-linked immunospot assay were tested again to confirm the specificity by using 15-mer peptides contained in the positive peptide pool. This time, each 15-mer peptide was tested separately (10 mM). One thousand T-clone cells were added per well in triplicate along with 1×10⁵ autologous Epstein-Barr Virus-transformed B-lymphoblastoid cell line cells and 20 U/ml of recombinant human IL-2. Otherwise, enzyme-linked immunospot assay was performed as described above.

### EXAMPLE 10

### Determining Whether the T-Cell Epitope is Naturally Processed

To assess whether the T-cell epitope being studied is endogenously processed as in the major histocompatibility complex class I pathway, an enzyme-linked immunospot assay was performed using the autologous Epstein-Barr Virus-transformed B-lymphoblastoid cell line cells (1×10⁵ cells per well) infected with recombinant vaccinia virus expressing the E6 protein, E7 protein or the wild-type virus, Western Reserve, at a multiplicity of infection of 5 in duplicate. One thousand T-clone cells were added per well. As a positive control for endogenous processing a known HPV 16 E7 epitope, a T-cell clone (27G6) previously shown to recognize an HPV 16 E7 79-87 (LEDLLMGTL; SEQ ID NO: 9) epitope restricted by an Human Leukocyte Antigen-B69 molecule (36) was incubated with autologous Epstein-Barr Virus-transformed B-lymphoblastoid cell line cells infected with E6 protein, E7 protein or wild type Western Reserve.

Another enzyme-linked immunospot assay was performed to assess whether the T-cell epitope of interest is being presented exogenously through an major histocompatibility complex class II pathway by pulsing recombinant E7 protein to autologous mature dendritic cells as described previously. Two thousand five hundred dendritic cells pulsed with recombinant E7 proteins were plated per well in duplicate. Peptide (HPV 16 E7 56-70; 10 mM)-pulsed dendritic cells were used as a positive control, and dendritic cells alone were included as a negative control. Fifty thousand autologous Epstein-Barr Virus-transformed B-lymphoblastoid cell line cells and 20 U/ml of recombinant human IL-2 were added to all wells.

### EXAMPLE 11

### Characterizing the Surface Phenotype of the T-Cell Clones

The T-cell clones for which peptide specificities were confirmed were analyzed for their surface phenotypes. The T-cell clones were stained with CD4PE/CD8-FITC cocktail, CD3-FITC/CD16-PE cocktail, and corresponding isotype controls (Caltag Laboratories, Burlingame, Calif.), and were analyzed using Coulter EPICS XL-MLC flow cytometer (Beckman Coulter, Fullerton, Calif.).

### EXAMPLE 12

### Identifying the Core Amino Acid Sequence of the CD4 T-Cell Epitope

A series of enzyme-linked immunospot assays were performed to define the core sequence of the novel CD4 T-cell epitope from subject 15-04. All peptides were used at a concentration of 10 mM, and 1×10³ cells from each T-cell clone along with 5×10⁴ or 1×10⁵ autologous Epstein-Barr Virus-transformed B-lymphoblastoid cell line cells were plated to each well in duplicate or triplicate. An enzyme-linked immunospot assay was first performed with 9-mer peptides overlapped by the central 8 amino acids that cover the HPV 16 E6 56-70 region. Since none of the 9-mer peptides was positive, another enzyme-linked immunospot assay was performed using 10-mer peptides overlapping by 9 amino acids in the same region. Subsequently, an enzyme-linked immunospot assay was repeated with two 10-mer peptides with the most numbers of spot forming units, 11-mer peptide encompassing these two 10-mers, and three 9-mers contained in the 11-mer peptide. Serial dilution of the two 10-mers, and the 11-mer was also performed to define the core sequence of the novel CD4 T-cell epitope.

### EXAMPLE 13

### Identifying the Restricting Human Leukocyte Antigen Molecule

The Human Leukocyte Antigen class II molecule presenting the novel CD4 T-cell epitope was determined using an enzyme-linked immunospot assay. Six allogeneic Epstein-Barr Virus-transformed B-lymphoblastoid cell line cells sharing one or more class II molecule(s) with the subject being studied (Human Leukocyte Antigen-DR15, -DR17, -DR51, -DR52, -DQ2, -DQ6) were used for the enzyme-linked immunospot assay (1×10³ T-clone cells were plated along with 1×10⁵ allogeneic Epstein-Barr Virus-transformed B-lymphoblastoid cell line cells per well in triplicate). The E7 56-70 peptide was used at a concentration of 10 mM. The Human Leukocyte Antigen type shared between the subject and the allogeneic Epstein-Barr Virus-transformed B-lymphoblastoid cell line cells with the largest number of spot-forming units was designated the restriction element if the results were corroborated with at least two allogeneic Epstein-Barr Virus-transformed B-lymphoblastoid cell line cells.

### EXAMPLE 14

### Human Leukocyte Antigen Typing

Human Leukocyte Antigen typing was performed at the University of Arkansas for Medical Sciences Human Leukocyte Antigen Laboratory by serological method or sequence-specific primers and polymerase chain reaction method.

### EXAMPLE 15

### Patterns of T-Cell Responses to HPV 16 E7 Protein in Vaccinated Subjects

Seventeen T-cell lines with at least one T-cell line from each subject were available to examine the presence of potential T-cell epitopes using enzyme-linked immunospot assay. The cell recovery of thawed T-cell lines was adequate (i.e., 1×10⁵ cells available per well in duplicates for peptide wells and media wells) for twelve T-cell lines from eight subjects [subject 5-02 time 3, subject 5-03 time 2 (HPV 18), subject 10-01 time 2, subject 10-02 times 1 and 2, subject 15-01 times 1 and 2, subject 15-02 times 2 and 3, subject 15-03 times 1 and 2, and subject 15-04 time 2]. As summarized in Table 3, enzyme-linked immunospot results from four HPV 16-positive subjects (5-02, 10-01, 15-03, 15-04) demonstrated the presence of potential T-cell epitopes. All of the four subjects showed a peak in the E7 46-70 region. For subjects 5-02, 10-01, and 15-03, this region represented the dominant peak, but the E7 61-85 region appeared to be the dominant one for subject 15-04. A subdominant T-cell response was also seen for subject 10-01 in the E7 76-98 region.

TABLE 3
Patterns of T-cell responses to HPV 16 E7 protein
in women with HPV 16⁺ cervical cancer treated
with dendritic cell immunotherapy

| | E7 region | | | | | |
|---|---|---|---|---|---|---|
| Subject | 1-25 | 16-40 | 31-55 | 46-70 | 61-85 | 76-98 |
| 5-02 [Time 3] | | | | X | | |
| 10-01 [Time 2] | | | | X | | X |
| 15-03 [Time 1] | | | | X | | |
| 15-04 [Time 2] | | | | X | X | |
| | | | | (58-68)^{†} | | |

Each region is covered by three 15-mer peptides overlapped by 10 central amino acids. X, the strongest T-cell response for a given subject; X, subdominant T-cell response for a given subject.
^{†}T-cell epitopes and their amino acid residues were characterized for this subject.

### EXAMPLE 16

### Isolation of T-Cell Clones from T-Cell Lines Containing Potential T-Cell Epitopes

The remaining cells from the T-cell line from subjects 15-03 and 15-04 were used to isolate antigen-specific T-cells based on interferon-g secretion. The T-cell line from subject 15-03 (6.5×10⁵ cells) were stimulated with peptides contained in the E7 46-70 region while that (1.3×10⁶ cells) of subject 15-04 were stimulated with peptides contained in the E7 46-70 and E7 61-85 regions. Approximately 500 or 1,100 interferon-g secreting cells were isolated from subjects 15-03 or 15-04 respectively, and serial dilution was performed for the isolation of T-cell clones. While 599 T-cell clones were isolated from subject 15-04, no T-cell clones grew for subject 15-03 and no further analysis was performed.

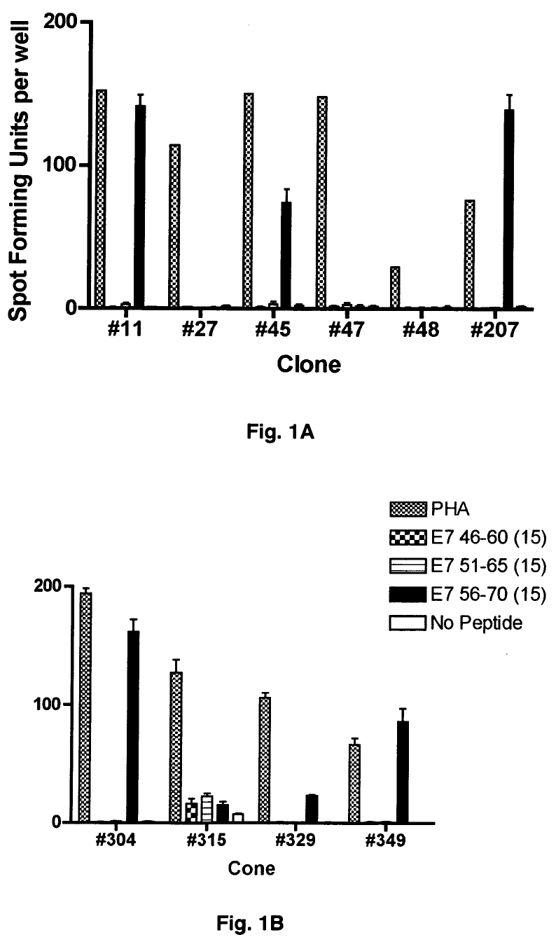

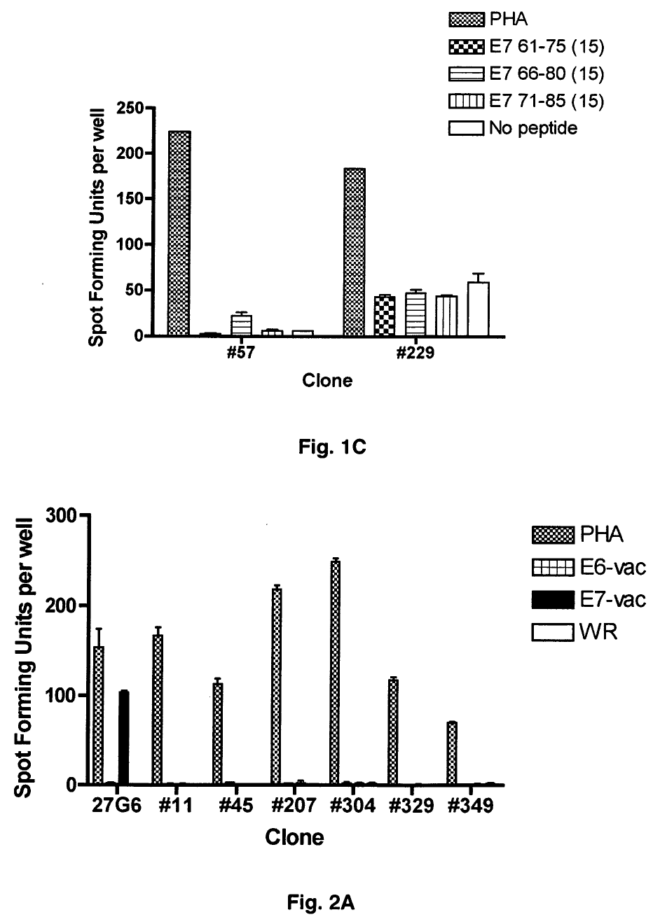

A random selection of 376 out of 599 expanded clones was screened with peptide pools covering E7 46-70 and E7 61-85, along with a no-peptide control. Ten T-cell clones were positive for the E7 46-70 pool, and two clones were positive for the E7 61-85 pool. Upon re-testing with individual peptides, six (#11, #45, #207, #304, #329, #349) of ten T-cell clones initially positive for the E7 46-70 pool were positive for the E7 56-70 peptide (FIGS. 1A-1B). The two T-cell clones initially positive for the E7 61-85 pool turned out to be false positive (FIG. 1C).

### EXAMPLE 17

### Examining the Mode of Antigen Presentation

### Major Histocompatibility Complex Class I Versus Class II Pathways

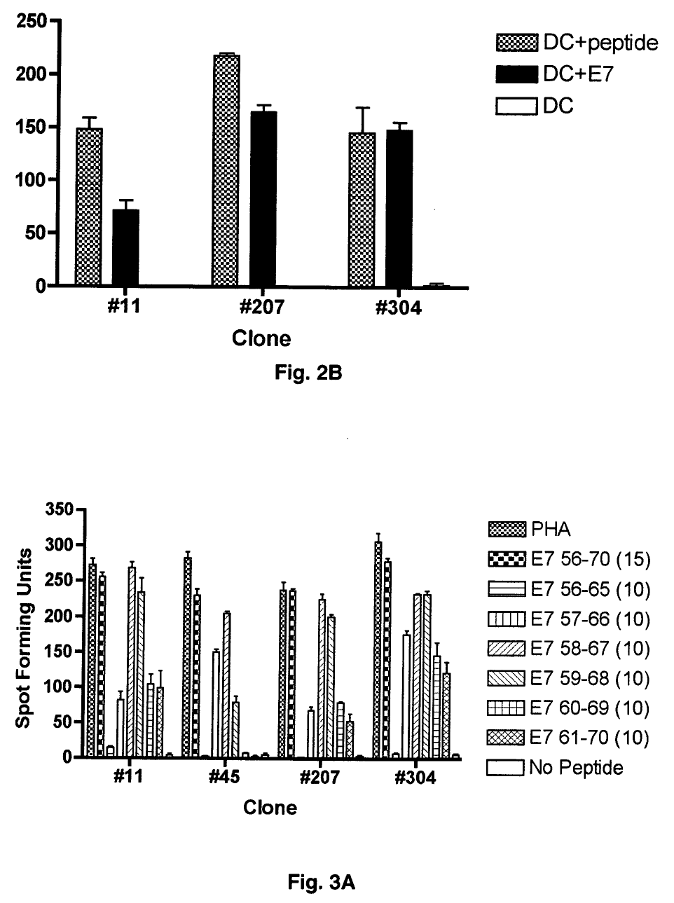

In order to confirm the specificity of the T-cell clones to the HPV 16 E7 protein and to examine the mode of T-cell antigen presentation, enzyme-linked immunospot assays were performed using autologous Epstein-Barr Virus-transformed B-lymphoblastoid cell line cells infected with recombinant vaccinia virus expressing the E7 protein (FIG. 2A) and autologous dendritic cells pulsed with the recombinant E7 protein, respectively (FIG. 2B). None of the T-cell clones tested (#11, #45, #207, #304, #329, #349) was positive when vaccinia virus expressing E6, vaccinia virus expressing E7, and wild type vaccinia virus infected autologous Epstein-Barr Virus-transformed B-lymphoblastoid cell line cells although a known HPV 16 E7 CD8 epitope was shown to be processed and recognized (FIG. 2A). On the other hand, all T-cell clones tested (#11, #207, #304) demonstrated interferon-γ secretion when autologous dendritic cells pulsed with E7 protein or E7 56-70 15-mer peptide was used (FIG. 2B). These results confirmed the specificity of the T-cell clones to HPV 16 E7 56-70 peptide or E7 protein but also suggested that the antigen needed to be presented exogenously and processed in a manner known for the Human Leukocyte Antigen Major Histocompatibility Complex class II antigen-processing pathway.

### EXAMPLE 18

### Analyzing the Surface Phenotype of Peptide-Specific T-Cell Clones

Flow cytometric analysis was performed to confirm the surface phenotype of E7 56-70 specific T-cell clones. The surface phenotypes of clones #11, #45, #207, #304, and #329 were CD3⁺ CD4⁺CD8⁻C16⁻ and that of clone #349 was CD3⁻CD4⁻CD8⁺CD16⁺. Clone #349 was not used for any further analysis.

### EXAMPLE 19

### Determining the Core Sequence of the Novel CD4 T-Cell Epitope

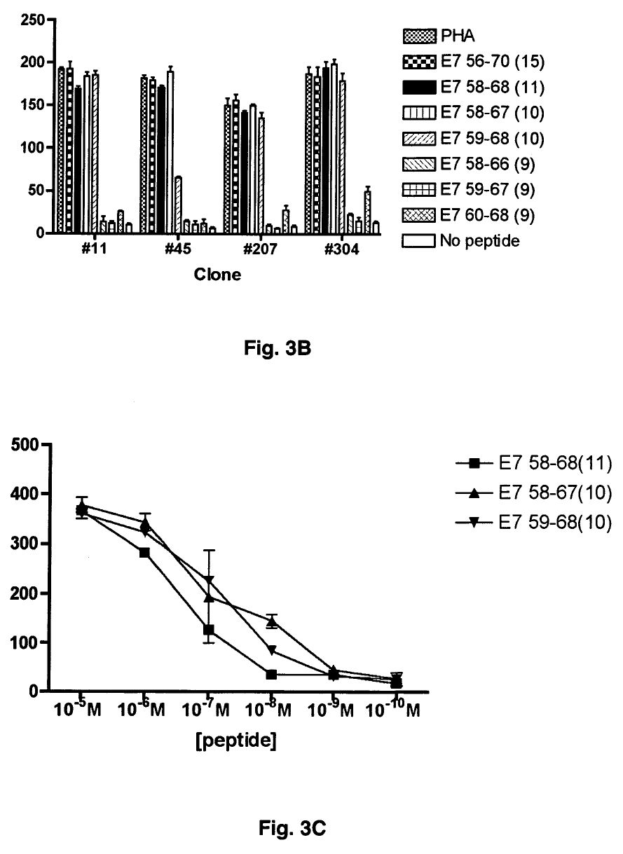

Because none of the T-cell clones tested was positive for the seven 9-mer peptides covering the HPV 16 E7 56-70 region, the enzyme-linked immunospot assay was repeated with 10-mer peptides overlapped by the central 9 amino acids (FIG. 3A). Among the six 10-mer peptides tested, the strongest response was seen for the E7 58-67 10-mer peptide, followed by the E7 59-68 10-mer peptide for three of the four T-cell clones tested (FIG. 3A). To better define the core sequence of this epitope, an enzyme-linked immunospot assay was repeated using an 11-mer peptide (E7 58-68) encompassing the two 10-mer peptides with the strongest responses along with E7 56-70 (15-mer), E7 58-67 (10-mer), E7 59-68 (10-mer), E7 58-66 (9-mer), E7 59-67 (9-mer), and E7 60-68 (9-mer) peptides. Based on experience defining CD8 T-cell epitopes, it was unusual that E7 58-67 (10-mer) and E7 59-68 (10-mer) had positive responses, but not E7 59-67 (9-mer) within the two 10-mer peptides. Therefore, new batches of 9-mer peptides were diluted for this experiment to exclude possibility of peptide degradation. Equally large numbers of spot-forming units were demonstrated with E7 56-70 (15-mer), E7 58-68 (11-mer), E7 58-67 (10-mer), E7 59-68 (10-mer), but the three 9-mer peptides were negative repeatedly (FIG. 3B). Furthermore, serially diluted E7 58-68 (11-mer), E7 58-67 (10-mer), E7 59-68 (10-mer) demonstrated equivalent numbers of spot forming units with all peptides tested for clone #45 and #207 (FIG. 3C). The experiment was repeated with serially diluted E7 58-68 (11-mer), E7 58-67 (10-mer), E7 59-68 (10-mer), and E7 46-70 (15-mer) for clone #207. The numbers of spot forming units were similar for all peptides tested.

### EXAMPLE 20

### The Restricting Human Leukocyte Antigen Molecule of the Novel CD4 T-Cell Epitope

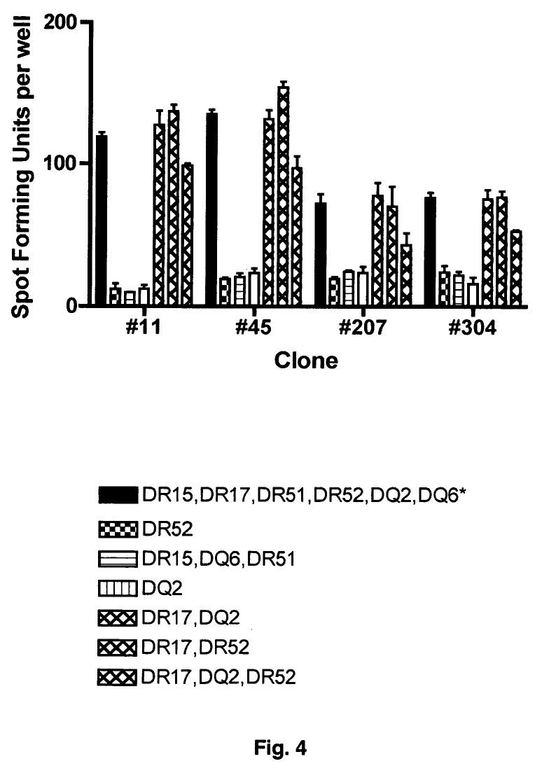

The allogeneic Epstein-Barr Virus-transformed B-lymphoblastoid cell line cells sharing one or more Human Leukocyte Antigen class II molecule(s) with subject 15-04 (Human Leukocyte Antigen-DR15, -DR17, -DR51, -DR52, -DQ2, -DQ6) were used to determine the restriction element. Results of an enzyme-linked immunospot assay showed more spot forming units were observed for the allogeneic Epstein-Barr Virus-transformed B-lymphoblastoid cell line cells, which express the Human Leukocyte Antigen-DR17 molecule but not with autologous Epstein-Barr Virus-transformed B-lymphoblastoid cell line cells expressing Human Leukocyte Antigen-DR15, -DQ2, -DQ6, -DR51, and -DR52 (FIG. 4). In all, restriction mapping enzyme-linked immunospot assay was performed three times. In all experiments, wells with Human Leukocyte Antigen-DR17 positive Epstein-Barr Virus-transformed B-lymphoblastoid cell line cells demonstrated a high number of spot forming units while the Human Leukocyte Antigen-DR17 negative autologous Epstein-Barr Virus-transformed B-lymphoblastoid cell line cells had a considerably lower number of spot forming units. Taken together, the restriction element was determined to be the Human Leukocyte Antigen-DR17 molecule for the novel HPV 16 E7 CD4 T-cell epitope.

The following references were cited herein:
1. World Health Organization, (1990) Global Estimates for Health Situationa Assessment and Projections 29-30.
2. Parkin et al. (1999) Intl J Cancer 80: 827-841.
3. Silverberg and Lubera, (1988) Cancer Journal for Clinicians 38; 5-22.
4. Munoz et al. (2003) N Engl J Med 348(6); 518-527.
5. zur Hausen, H. (1996) Biochim Biophys Acta 1288:F55-78.
6. zur Hausen, H. (1999) Semin Cancer Biol 9:405-411.
7. Beaudenon et al. (1986) Nature 321(6067); 246-249.
8. Crum et al. (1985) J Virol 54(3); 675-681.
9. Reid et al. (1987) Obstet Gynecol Clin North Am 14(2):407-429.
10. Lorincz et al. (1986) J Virol 58:225-229.
11. Lorincz et al. (1987) Virol 159:187-190.
12. Fuchs et al. (1988) Int J Cancer 41(1) 41-45.
13. Koutsky et al. (1992) N Engl J Med 327(18): 1272-1278.
14. Richart et al. (1969) Am J Obstet Gynecol 105: 383-393.
15. Nash et al. (1987) Obstet Gynecol 69: 160-162.
16. Campion et al. (1986) Lancet 2: 236-240.
17. Santin et al. (2002) N Engl J Med 346(22): 1752-1753.
18. Greenberg, P. D. (1991) Adv Immunol 49:281-355.
19. Ossendorp et al. (1998) J Exp Med 187:693-702.
20. Romerdahl et al. (1988) Cancer Res 48:2325-2328.
21. Schild et al. (1987) EurJ Immunol 17:1863-1866.
22. Bontkes et al. (1997) Br J Cancer 76(10): 1353-1360.
23. Bennett et al. (1998) Nature 393:478-480.
24. Ridge et al. (1998) Nature 393:474-478.
25. Schoenberger et al. (1998) Nature 393:480-483.
26. Snijders et al. (1998) Int Immunol 10:1593-1598.
27. Romani et al. (1996) J Immunol Methods 196:137-151.
28. Osada et al. (2006) Int Rev Immunol 25:377-413.
29. Ferrara et al. (2003) J Cancer Res Clin Oncol 129:521-530.
30. Santin et al. (2006) Gynecol Oncol 100:469-478.
31. van der Burg et al. (2001) International Journal of Cancer 91:612-618.
32. Okubo et al. (2004) Jour of Obstetrics and Gynaecology Research 30:120-129.
33. Peng et al. (2007) Clin Cancer Res 13:2479-2487.
34. Gallagher and Man S, (2007) J Gen Virol 88:1470-1478.
35. Rickinson and Moss, (1997) Ann Rev Immunol 15:405-431.
36. Nakagawa et al. (2004) Clinical and Diagnostic Lab Immunology 11:889-896.
37. Nakagawa et al. (2005) Clin Diagn Lab Immunol 12:1003-1005.
38. Kaul et al., (2001) J Clin Invest 107:1303-1310.
39. Nakagawa et al. (2007) J Virol 81:1412-1423.
40. Walls, E. V. and Crawford L H, In: Klaus G G B, ed. Lymphocytes: A practical approach. Oxford, U.K., IRL Press, 1987
41. Nakagawa et al. (1997) J Infect Dis 175:927-931.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of determining immunodominant T cell epitopes within a protein expressed in an individual, comprising:
administering autologous dendritic cells pulsed with a recombinant protein to said individual;
establishing T-cell lines from the individual;
incubating said T cell lines with peptides representative of the protein;
measuring the specific T cell response in the incubated cells; and
identifying peptides that induce T cell response, wherein sequence of the peptide corresponds to a region within the protein, thereby determining the immunodominant T cell epitopes within the protein in the individual.

2. The method of claim 1, further comprising:
determining an amino acid sequence of the immunodominant T cell epitopes identified in claim 1.

3. The method of claim 1, wherein the individual is diagnosed with a pathophysiological condition, is in remission or is diagnosed with a precursor of the pathophysiological condition.

4. The method of claim 3, wherein the pathophysiological condition is a neoplastic disease or disorder, an autoimmune disease or disorder or a pathogen-related infection or disease.

5. The method of claim 4, wherein the neoplastic disease or disorder is a Human Papilloma virus infection, atypical squamous cells of undetermined significance, squamous intraepithelial lesion, cervical intraepithelial lesion, cervical cancer, prostate cancer, ovarian cancer, vulvar cancer, anal cancer, head cancer, neck cancer or other types of cancers.

6. The method of claim 1, wherein the T cell epitopes determined are CD4 T cell epitopes or CD8 T cell epitopes.

7. The method of claim 1, wherein the peptides that comprise the immunodominant T cell epitope in Human Papilloma virus protein have amino acid sequence of SEQ ID NO: 1, SEQ ID NO: 2, SEQ ID NO: 3, SEQ ID NO: 4; SEQ ID NO: 5; SEQ ID NO: 6; SEQ ID NO:7 or SEQ ID NO: 8.

8. The method of claim 1 wherein the peptides that comprise the immunodominant T cell epitope in Human Papilloma virus protein have at least an 80% similarity and up to and including a 90% similarity in amino acid sequence to SEQ ID NO: 1, SEQ ID NO: 2, SEQ ID NO: 3, SEQ ID NO: 4; SEQ ID NO: 5; SEQ ID NO: 6; SEQ ID NO: 7 or SEQ ID NO: 8.

9. The method of claim 1, wherein the immunodominant T cell epitopes are contained in a 25-amino acid residue long peptide.

10. The method of claim 9, wherein the peptide comprising the immunodominant T cell epitopes in Human Papilloma virus protein has an amino acid sequence of SEQ ID NO. 8.

11. The method of claim 7, wherein the immunodominant T cell epitope is 11 amino acid residues long peptide.

12. The method of claim 11, wherein the immunodominant T cell epitope in the Human Papilloma virus protein has an amino acid sequence of SEQ ID NO. 7.

13. A method of immunotherapy directed towards a protein in an individual, comprising:
isolating immune cells from the individual;
incubating the isolated immune cells with peptides comprising one or more than one immunodominant T cell epitopes identified using the method of claim 1; and
transferring said incubated immune cells back to the individual, wherein the immune cells produce a specific immune response in the individual, thereby generating immunotherapy targeted towards the protein in the individual.

14. The method of claim 13, wherein the protein is Human Papilloma virus E6 or E7 protein.

15. The method of claim 14, wherein the immune cells are T cells or dendritic cells.

16. The method of claim 14, wherein the individual has a positive Human Papilloma virus DNA test, an abnormal pap smear results, has been diagnosed with a precursor of cervical cancer, has been diagnosed with cervical cancer or is suspected or at risk of suffering from cervical cancer.

17. The method of claim 13 wherein the peptide comprising one or more immunodominant T cell epitopes is a synthetic peptide having a sequence shown in SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

18. The method of claim 17 wherein the synthetic peptide comprising the immunodominant T cell epitope in Human Papilloma virus protein has at least an 80% similarity and up to and including a 90% similarity in amino acid sequence to SEQ ID NO: 1, SEQ ID NO: 2, SEQ ID NO: 3, SEQ ID NO: 4; SEQ ID NO: 5; SEQ ID NO: 6; SEQ ID NO: 7 or SEQ ID NO: 8.

19. An immunogenic composition comprising one or more peptides having a sequence shown in SEQ ID NO: 1, SEQ ID NO: 2, SEQ ID NO: 3, SEQ ID NO: 4; SEQ ID NO: 5; SEQ ID NO: 6; SEQ ID NO: 7 or SEQ ID NO: 8 and an immunologically acceptable adjuvant.

20. The immunogenic composition of claim 19, wherein the peptide sequence has at least an 80% similarity and up to and including a 90% similarity in amino acid sequence to SEQ ID NO: 1, SEQ ID NO: 2, SEQ ID NO: 3, SEQ ID NO: 4; SEQ ID NO: 5; SEQ ID NO: 6; SEQ ID NO: 7 or SEQ ID NO: 8.

21. The immunogenic composition of claim 19, wherein the adjuvant is Candida, mumps or Trichophyton.

22. The immunogenic composition of claim 19, wherein the sequence(s) is expressed in a recombinant viral vector, in a plasmid or as a synthetic peptide.

23. A method of preventing or treating a pathophysiological condition involving expression of protein in an individual, comprising:
administering an immunologically effective amount of the immunogenic composition of claim 19 to an individual, wherein the composition activates a specific immune response in the individual, thereby preventing or treating the pathophysiological condition in the individual.

24. The method of claim 19, wherein the pathophysiological condition the individual has been diagnosed with is a Human Papilloma virus infection, a precursor of cancer, is cancer, or is suspected or at risk of suffering from cancer.

25. The method of claim 24, wherein the cancer is Human Papilloma Virus positive.

26. A synthetic peptide comprising one or more peptides having a sequence shown in SEQ ID NO: 1, SEQ ID NO: 2, SEQ ID NO: 3, SEQ ID NO: 4; SEQ ID NO: 5; SEQ ID NO: 6; SEQ ID NO: 7 or SEQ ID NO: 8 or one or more peptides having at least an 80% similarity and up to and including a 90% similarity in amino acid sequence thereto.
